(19) United States
(12) Patent Application Publication
(10) Pub. No.: US 2015/0133557 A1
(43) Pub. Date: May 14, 2015

(54) ALDITOL FREE AND SACCHARIDE FREE STORAGE-STABLE THYROID HORMONE ACTIVE DRUG FORMULATIONS AND METHODS FOR THEIR PRODUCTION

(71) Applicants: Nilesh Parikh, Irvine, CA (US); William Hite, Winchester, CA (US)

(72) Inventors: Nilesh Parikh, Irvine, CA (US); William Hite, Winchester, CA (US)

(21) Appl. No.: 14/080,646

(22) Filed: Nov. 14, 2013

(51) Int. Cl. A61K 47/10; A61K 31/197

(52) U.S. Cl. CPC A61K 47/10; A61K 31/197
USPC 514/567

(57) ABSTRACT

Embodiments of the present invention provide pharmaceutical compositions in unit dosage form that possess a therapeutically effective amount of a levothyroxine sodium; an antioxidant in an amount sufficient to inhibit oxidation of the levothyroxine sodium in the unit dosage form; an amount of a calcium phosphate sufficient to facilitate a processing of the levothyroxine into the unit dosage form, and two or more excipients selected from the group consisting of a filler, a binder, a glidant, a lubricant, a disintegrant, and a surfactant. In such embodiments, the compositions are free of added alditol, added monosaccharide, added disaccharide, and added oligosaccharide and are storage stable.

### FIELD OF THE INVENTIONS

Embodiments of the invention relate to methods of preparing storage-stable pharmaceutical compositions of thyroid hormone and compositions made by these methods.

### BACKGROUND OF THE INVENTIONS

Thyroid hormone active drugs are known for both therapeutic and prophylactic treatment of thyroid disorders. For example, levothyroxine sodium is prescribed for thyroid hormone replacement therapy in cases of reduced or absent thyroid function in ailments such as myxedema, cretinism, and obesity. See, for example, Post and Warren in Analytical Profiles of Drug Substances, Vol. 5, Florey (ed.); Academic Press, New York (1976), pp. 226-281. Levothyroxine sodium is quite unstable, hygroscopic and degrades rapidly when subjected to high humidity, light or high temperature. See, for example, Won, Pharm. Res. 9(1):131-137, 1992. Because of the physico-chemico properties of the drug, many levothyroxine sodium formulations have short stability duration, worsened under conditions of high humidity and temperature. Tablets may decompose approximately 1 percent per month. Gupta et. al., J. Clin. Pharm. Ther. 15:331-335, 1990. The stability problem has been so widespread that some drug companies marketing levothyroxine sodium tablets have been forced to recall various batches due to lack of stability.

Formulations containing levothyroxine sodium have been known since the late 1950s. There have been attempts to develop more stable dosage formulations of levothyroxine sodium. For example, U.S. Pat. No. 5,635,209 discloses levothyroxine sodium in combination with potassium iodide as part of a stabilizing excipient. In the manufacture of this formulation, levothyroxine sodium was first mixed with microcrystalline cellulose, and then added to a dried granulation of potassium iodide and microcrystalline cellulose. The formulation purportedly provided increased active drug potency over a three month period in comparison to then commercially available formulations.

U.S. Pat. No. 5,225,204 teaches a complex of levothyroxine sodium and a cellulose, polyvinylpyrrolidone or poloxamer. The formulation may be prepared by dissolving the drug complex in a polar organic solvent, adding a cellulose carrier to the liquid, and drying the resulting mixture to obtain a complex of levothyroxine sodium and polyvinylpyrrolidone or poloxamer adsorbed on the cellulose carrier. Tests of such combinations yielded stability results at best equal to commercially available preparations such as those described in U.S. Pat. No. 5,955,105, and in some cases substantially worse. The inventors of this stabilized composition teach one of skill in the art away from the use of carbohydrates in levothyroxine sodium formulations, stating that instability of the dosage form was the result of an interaction between the active drug substance and carbohydrate excipients.

U.S. Pat. No. 5,955,105 teaches that levothyroxine is relatively stable in pure form and that the instability of levothyroxine is due to its interaction with particular excipients. The patent teaches that thyroid hormones, particularly levothyroxine sodium, are compatible with carbohydrates, such as starch and maltodextrin, but incompatible with lactose, glucose and sucrose. The patent teaches a formula for direct compression levothyroxine sodium dosage forms that contains a soluble polysaccharide, designed to eliminate the interaction between the drug and other excipients, and carbohydrate having a molecular weight greater than 500.

U.S. Pat Nos. 7,195,779 and 7,052,717 teach storage-stable pharmaceutical compositions of thyroid hormones, such as levothyroxine sodium, are achieved by blending the active ingredient with stabilizing amounts of mannitol and sucrose, or mannitol, sucrose, and antioxidant butylated hydroxyanisole (BHA), to form a granulation intermediate. The patents teach that a stabilizing effect is achieved for levothyroxine sodium in its formulations due to the presence of the mannitol and sucrose, or mannitol sucrose, and BHA both at an early stage of manufacture and in the final dosage form.

### SUMMARY OF THE INVENTIONS

Embodiments of the present invention provide pharmaceutical compositions in unit dosage form that possess a therapeutically effective amount of a levothyroxine sodium; an antioxidant in an amount sufficient to inhibit oxidation of the levothyroxine sodium in the unit dosage form; an amount of a calcium phosphate sufficient to facilitate a processing of the levothyroxine into the unit dosage form, and two or more excipients selected from the group consisting of a filler, a binder, glidant, a lubricant, a disintegrant, and a surfactant. In such embodiments, the compositions are free of added alditol, added monosaccharide, added disaccharide, and added oligosaccharide and are storage stable.

In some embodiments, the antioxidant is one or more of a butylated hydroxyanisole, a vitamin E (alpha tocopherol), a butylated hydroxytoluene an ascorbic acid, a sodium ascorbate a sodium bisulfate, and a sodium metabisulfite.

In some embodiments, the calcium phosphate is a dicalcium phosphate.

In some embodiments, the filler and the binder are each one or more of a polyvinylpyrrolidone, sodium citrate, an alkaline inorganic salt, an alginate, a gelatin, a microcrystalline cellulose, a chitosan, a kaolin, a magnesium carbonate, a calcium carbonate, an acacia, a cellulose, a gelatin, a starch, a polyethylene glycol, a polyvinyl alcohol, and a polymethacrylate.

In some embodiments, the glidant and the lubricant are each at one or more of a talc, a silica, a fumed silica, and a colloidal silicon dioxide, and wherein the lubricant is at least one member selected from the group consisting of a talc, a calcium stearate, a sodium stearyl fumarate, a stearic acid, a magnesium stearate, a solid polyethylene glycol, a cocoa butter, a hydrogenated vegetable oil, a mineral oil, a sodium lauryl sulfate, a glyceryl palmitostearate, and a glycetyl behenate.

In some embodiments, the disintegrant is at least one member selected from the group consisting of a crospovidone, a crosscarmelose sodium, a sodium starch glycolate, a partially gelatinized starch, and a polacrilin potassium.

In some embodiments the surfactant is one or more of a polysorbate, a sodium lauryl sulfate, a lauryl dimethyl amine oxide, a cetyltrimethylammonium bromide, a polyethoxylated alcohol, a polyoxyethylene sorbitan, octoxynol, a n,n-dimethyldoclecylamine-n-oxide, a hexadecyltrimethylammonium bromide, a polyoxyl 10 lauryl ether, a BRIJ 721, a bile salt, a polyoxyl castor oil, a nonylphenol ethoxylate, a cyclodextrin, a lecithin, and a methylbenzethonium chloride.

Certain embodiments of the invention provide pharmaceutical compositions, in unit dosage form, that possess levothyroxine sodium in an amount of 25 μg, 50 μg, 75 μg, 88 μg, 100 μg, 112 μg, 137 μg, 150 μg, 175 μg, 200 μg, or 300 μg, from 0.001% to 1% w/w of an antioxidant that is a butylated hydroxyanisole a vitamin E (alpha tocopherol), a butylated hydroxytoluene, an ascorbic add, a sodium ascorbate, a sodium bisulfate, and a sodium metabisulfite, or a combination thereof, from 25% to 95% w/w of a microcrystalline cellulose; and from 35% to 45% w/w of a calcium phosphate. In such embodiments, the compositions are free of added alditol, added monosaccharide added disaccharide, and added oligosaccharide, and are storage stable.

In some embodiments, the compositions further possess from 0.10% to 3.00% w/w of a polyvinylpyrrolidone. In some embodiments, the polyvinylpyrrolidone is a low peroxide K30 polyvinylpyrrolidone.

In some embodiments, the compositions further possess from 0.1% to 5.00% w/w of each of a glidant and a lubricant that are one or more of a talc, a silica, a fumed silica, and a colloidal silicon dioxide, and wherein the lubricant is at least one member selected from the group consisting of a talc, a calcium stearate, a sodium stearyl fumarate, a stearic acid, a magnesium stearate, a solid polyethylene glycol, a cocoa butter, a hydrogenated vegetable oil, a mineral oil, a sodium lauryl sulfate, a glyceryl palmitostearate, and a glyceryl behenate.

In some embodiments, the compositions further possess from 0.01% to 5.0% of a surfactant that are one or more of a polysorbate, a sodium lauryl sulfate, a lauryl dimethyl amine oxide, a cetyltrimethylammonium bromide, a polyethoxylated alcohol, a polyoxyethylene sorbitan, an octoxynol, a n,n-dimethyldodecylamine-n-oxide, hexadecyltrimethylammonium bromide, a polyoxyl 10 lauryl ether, a BRIJ 721, a bile salt, a polyoxyl castor oil, a nonylphenol ethoxylate, a cyclodextrin, a lecithin, and a methylbenzethonium chloride.

### DETAILED DESCRIPTION OF THE INVENTIONS

The prior art teaches that the combinations of alditol and mono-, di-, or oligo-saccharide and alditol, mono-, di-, or oligo-saccharide, and BHA provide stable granulation intermediates of levothyroxine, to which additional excipients may be added to form unit dosage form oral pharmaceutical compositions that maintain a predictable dosage of active levothyroxine for a substantial period of time. The compositions of the present invention provide pharmaceutical compositions in unit dosage form that possess a therapeutically effective amount of levothyroxine sodium; an antioxidant in an amount sufficient to stabilize the levothyroxine sodium against oxidation; and amounts of a calcium phosphate and one or more of a filler, a binder, and a lubricant sufficient to facilitate processing the compositions into the unit dosage form. Such compositions are free of added alditol, free of added monosaccharide, free of added disaccharide, and free of added oligosaccharide. The unit dosage form oral pharmaceutical compositions of the invention have unexpectedly excellent storage-stability properties. Accordingly, the invention provides a stable dosage form in which the dosage of thyroxine active drug such as levothyroxine sodium, is surprisingly maintained at a predictable level for a substantial period of time.

In some embodiments, methods of making the unit dosage form oral pharmaceutical compositions of the invention include preparing a granulation intermediate containing the thyroxine active drug substance, BHA and a monosaccharide, a disaccharide, and/or an oligosaccharide, to which pharmaceutically acceptable excipients are added. This intermediate can be used to produce stable formulations of any natural or synthetic thyroid hormone replacement drug. Therefore, although the following description and examples refer to compositions and methods using levothyroxine sodium, the hormone embodiments of the invention encompass other thyroid hormone medications of the general formula I:

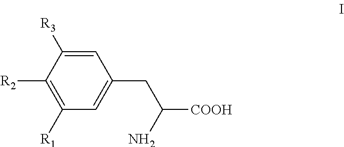

wherein R₁ and R₃ may be the same or different and are selected from hydrogen; halogen; alkyl; aryl; cycloalkyl; heterocycloalkyl; amide; alcohol; acid; ester; ether; acyl; alkenyl; and alkynyl; wherein R₂ is

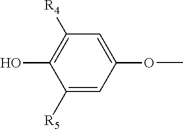

wherein R₄ and R₅ may be the same or different and are selected from hydrogen; halogen; alkyl; aryl; cycloalkyl; heterocycloalkyl; amide; alcohol; add; ester; ether; acyl; alkenyl; and alkynyl. The medication can be in the form of a free add, a free base, an organic salt, an inorganic salt, or a hydrate. Liothyronine is an example of a drug encompassed by the above-mentioned general formula.

In certain embodiments, stabilized pharmaceutical compositions are produced by blending the active ingredient with microcrystalline cellulose and granulating with an antioxidant to form a granulation intermediate. Further pharmaceutical excipients are generally added to produce final oral dosage forms, such as tablets, capsules, powders, or sachets, though such addition is optional. Useful granulation solvents include water and ethanol.

In certain embodiments, antioxidants that have low water solubility, such as BHA or BHT, may be added to a solvent in which they are reasonably soluble, such as alcohol, glycerin, and propylene glycol, and then used in preparing a granulation intermediate. In some embodiments, water may be added to such a water-insoluble, antioxidant-solvent solution to form a solution (e.g., about 10-90% water) for use in preparing a granulation intermediate. In some embodiments, a polyvinylpyrrolidone is added to the antioxidant solution.

In certain embodiments, formulations according to the present invention are made according to the following general steps. The active thyroxine ingredient (e.g., levothyroxine sodium) is blended with a microcrystalline cellulose and optionally an antioxidant, such as BHA or BHT, to form a pre-blend. Granulation intermediates are produced by making a wet granulation of the active ingredient with: i. the antioxidant-solvent solution or the antioxidant-solvent solution and polyvinylpyrrolidone mix, and ii. the active thyroxine ingredient microcrystalline cellulose pre-blend. At least one of the pre-blend and the solution used for preparing the granulation intermediate include(s) an antioxidant. The levothyroxine sodium is thus first blended with the microcrystalline cellulose, and then further excipients dicalcium phosphate and/or polyvinylpyrrolidone as binder) may be incorporated into the granulation intermediate, but need not be added until the active ingredient is intimately mixed with the microcrystalline cellulose and antioxidant. Therefore, dicalcium phosphate and/or other diluent functions as a granulation aid and a compression enhancer (for tablet or capsule formulations) and not as a specific carrier for the thyroxine active drug,

In some embodiments, the wet granulation mix is dried milled and optionally further blended. The granulation intermediate then may be stored or directly mixed with further ingredients to form a composition suitable for compression into tablets, into capsules or sachets, or dissolved or suspended to form a liquid dosage form.

Pharmaceutical compositions of the invention may be prepared for administration orally, rectally, vaginally, transmucosally, transdermally parenterally, subcutaneously, and intramuscularly. Pharmaceutically acceptable excipients suitable for use in such compositions include, but are not limited to adjuvants, preservatives, buffers, antioxidants, fillers, extenders, carriers, binders, diluents, disintegrants, glidants lubricants, surfactants, wetting agents, surface active agents, suspending agents, and solvents. Compounds such as dyes, colorants, sweeteners, flavorings, perfuming agents, and taste-masking agents also may be included in formulations according to this invention. In addition, other active ingredients may he included to produce a dual or multiple ingredient medication.

Exemplary disintegrants may be selected from known pharmaceutical excipients such as, for example, crospovidone, crosscarmelose sodium, sodium starch glycolate, partially gelatinized startches, polacrilin potassium.

Exemplary surfactants may be selected from known pharmaceutical excipients such as for example, stearic acid or other fatty acids, polysorbates, sodium dodecyl sulfate (sodium lauryl sulfate), lauryl dimethyl amine oxide, cetyltrimethylammonium bromide, polyethoxylated alcohols, polyoxyethylene sorbitan, octoxynol (TRITON X100™), n,n-dimethyldodecylamine-n-oxide, hexadecyltrimethylammonium bromide (HTAB), polyoxyl 10 lauryl ether, BRIJ 721, bile salts (e.g., sodium deoxycholate, sodium cholate), polyoxyl castor oil (CREMOPHOR™), nonylphenol ethoxylate (TERGITOL™), cyclodextrins, lecithin, and methylbenzethonium chloride (HYAMINE™).

Exemplary lubricants and/or glidants may be selected from known pharmaceutical excipients such as, for example, talc, calcium stearate, sodium stearyl fumarate stearic acid, magnesium stearate, solid polyethylene glycols, cocoa butter, hydrogenated vegetable oil, mineral oil, sodium lauryl sulfate, glyceryl palmitostearate, and a glyceryl behenate.

Exemplary binders and/or fillers may be selected from known pharmaceutical excipients such as, for example, polyvinylpyrrolidone, sodium citrate, dicalcium phosphate, alkaline inorganic salts, alginates, gelatins, microcrystalline cellulose, chitosan, kaolin, magnesium carbonate, calcium carbonate, acacia, methyl cellulose, liquid glucose tragacanth, ethyl cellulose, gelatin, hydroxypropylmethyl cellulose (HPMC), starch paste, hydroxypropyl cellulose, starch, pregelatinized starch, sodium carboxymethyl cellulose, alginic acid, polyvinyl pyrrolidone (PVP), cellulose, polyethylene glycol (PEG), polyvinyl alcohols, and polymethacrylates.

Solid dosage forms which may be prepared according to this invention include tablets, capsules, rectal or vaginal suppositories, pills, dragees, lozenges, granules, beads, microspheres, pellets and powders, or any combination thereof.

The preferred active ingredient in the formulations of this invention is levothyroxine sodium. Therapeutically effective dosage amounts for this drug generally range from about 0.1 μg to about 5000 μg and are most preferably from about 25 μg to about 300 μg. Exemplary dosages therefore include, butane not limited to 20 μg, 25 μg, 50 μg, 75 μg, 88 μg, 100 μg 125 μg, 150 μg, 175 μg and 300 μg.

Antioxidant percentages, by weight of dosage form include, but are not limited to, from about 0.001% to about 5%, such as about 0.001%, about 0.005%, about 0.01%, about 0.05%, about 0.1%, about 0.5%, about 1%, about 1.5%, about 2%, about 2.5%, about 3%, about 3.5%, about 4%, about 4.5%, about 5%, and ranges therebetween.

Binder percentages, by weight of dosage form include, but are not limited to, from about 0.001% to about 10% such as about 0.001%, about 0.005%, about 0.01%, about 0.05%, about 0.1%, about 0.5%, about 1%, about 1.5%, about 2%, about 2.5%, about 3%, about 3.5%, about 4%, about 4.5%, about 5%, about 6%, about 7%, about 8%, about 9%, about 10% and ranges therebetween.

Glidant percentages, by weight of dosage form include, but are not limited to, from about 0.1% to about 10% such as about 0.1%, about 0.5%, about 1% about 1.5%, about 2%, about 2.5%, about 3%, about 3.5%, about 4%, about 4.5%, about 5%, about 6%, about 7%, about 8%, about 9%, about 10% and ranges therebetween.

Surfactant percentages, by weight of dosage form include, but are not limited to, from about 0.001% to about 5%, such as about 0.001%, about 0.005%, about 0.01%, about 0.05%, about 0.1%, about 0.5%, about 1%, about 1.5%, about 2%, about 2.5%, about 3%, about 3.5%, about 4%, about 4.5%, about 5%, and ranges therebetween.

Lubricant percentages, by weight of dosage form include, but are not limited to, from about 0.01% to about 10% such as about 0.01%, about 0.05%, about 0.1%, about 0.5%, about 1%, about 1.5%, about 2%, about 2.5%, about 3%, about 3.5%, about 4%, about 4.5%, about 5%, about 6%, about 7%, about 8%, about 9%, about 10% and ranges therebetween.

Disintegrant percentages, by weight of dosage form include, but are not limited to, from about 0.01% to about 1.0% such as about 0.01%, about 0.05%, about 0.1%, about 0.5%, about 1%, about 1.5%, about 2%, about 2.5%, about 3%, about 3.5%, about 4%, about 4.5%, about 5%, about 6%, about 7%, about 8%, about 9%, about 10% and ranges therebetween.

After the solid ingredients of the formulation are blended, the stabilized drug preparation preferably is compressed into tablets. Alternatively, the preparation may be used to fill capsules such as hard gelatin capsules or used to prepare any other convenient solid dosage form. Compositions according to the invention may be stored in the form of powders, granulates, intermediates, suspensions, or solutions prior to addition of additional desired pharmaceutical excipients for the production of final liquid dosage forms, such as solutions, syrups, suspensions, emulsions and the like.

The following examples further illustrate the invention and are not to be construed to limit the claims in any manner. Levothyroxine sodium tablets having the formulation set forth in Example 1 were prepared by the process set forth in Example 2.

### EXAMPLE 1

TABLE 1
Levothyroxine 25 μg Tablets

| Component | Amount in mg/tablet |
|---|---|
| Levothyroxine sodium (anhydrous basis) | 0.025 |
| Microcrystalline cellulose | 73.07 |
| Microcrystalline cellulose | 9.4 |
| Polyvinylpyrrolidone | 1.50 |
| Butylated hydroxyanisole | 0.015 |
| Dicalcium phosphate | 43.25 |
| FD & C yellow 6 aluminum lake | 0.39 |
| Colloidal silicon dioxide | 0.35 |
| Sodium lauryl sulfate | 0.12 |
| Magnesium stearate | 1.88 |
| Purified water* | qs |
| Ethanol* | qs |

Total tablet weight = 130 mg
*removed to trace amounts during processing

### EXAMPLE 2

The levothyroxine sodium tablets set forth in Example 1 were made by the following process.

Step 1—dry mixing. Microcrystalline cellulose and levothyroxine sodium, each previously sifted through a 40# sieve, were sequentially added to a granulator under conditions of impeller slow and chopper off and approximately 35% occupancy.

Step 2—granulating solution preparation. BHA and PVP were made into granulating solution(s).

Step 3—granulation. The granulation solution of step 2 was slowly added to the mixture formed in step 1 to form a wet granulation mass.

Step 4—drying. The wet mass of step 3 was dried in a fluid bed dryer at 40° C.±5° C. until loss on drying of no more than 4% w/w was achieved.

Step 5—milling and sifting. The dried granules of step 4 were mixed through a 0.5 mm screen and sifted through a 200# sieve.

Step 6—blending. The milled and sifted granules of step 5 were mixed with dicalcium phosphate that had been sifted through a #30 sieve in a contra blender at approximately 60% occupancy.

Step 7—color premix and blending. Additional microcrystalline cellulose was blended into the blend obtained in step 6 in a contra blender at approximately 60% occupancy. A color premix composed of microcrystalline cellulose, color 200#, colloidal silicon dioxide, sodium lauryl sulfate, and magnesium stearate was made and sifted through a 60# sieve.

Step 8—lubrication. The blend obtained in step 7 and the color premix made in step 7 were blended in a contra blender at approximately 60% occupancy.

Step 9—compression. The blend obtained in step 8 was compressed into tablets using a rotary compression machine.

### EXAMPLE 3

Table 2 reports stability data for the levothyroxine sodium tablets having the formulation set forth in Example 1 and prepared by the process set forth in Example 2. The tablets were stored for 5 days at 25° C//60% relative humidity (RH), 40° C./75% RH and 60° C./ambient RH in a suitable pharmaceutical container. Samples of these tablets were analyzed for drug potency using a stability indicating HPLC assay method. Evaluation of the potency for these tablets (i.e., percentage label claim of levothyroxine sodium in the tablet) after five days storage demonstrates that the formulation described in Example 1 yields a product which demonstrates good stability at all temperatures at or below 60° C.

TABLE 2

| Temperature/Relative Humidity (RH) | Tablet Potency |
|---|---|
| 5 days at 25° C./60% RH | 101.9 |
| 5 days at 40° C./75% RH | 101.3 |
| 5 days at 60° C./Ambient RH | 95.4 |

Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A pharmaceutical composition, in unit dosage form, that comprises: a therapeutically effective amount of a levothyroxine sodium; an antioxidant in an amount sufficient to inhibit oxidation of the levothyroxine sodium in the unit dosage form; an amount of a calcium phosphate sufficient to facilitate a processing of the levothyroxine into the unit dosage form, and two or more excipients selected from the group consisting of a filler, a binder, a glidant, a lubricant, a disintegrant, and a surfactant, wherein the composition is:
free of: added alditol, added monosaccharide, added disaccharide, and added oligosaccharide, and
storage stable.

2. The composition of claim 1, wherein the antioxidant is at least one member selected from the group consisting of a butylated hydroxyanisole, a vitamin E (alpha tocopherol), a butylated hydroxytoluene, an ascorbic acid, a sodium ascorbate, a sodium bisulfate, and a sodium metabisulfite.

3. The composition of claim 1, wherein the antioxidant is a butylated hydroxytoluene.

4. The composition of claim 1, wherein the calcium phosphate is a dicalcium phosphate.

5. The composition of claim 1, wherein the composition comprises the filler and the binder, and wherein the filler and the binder are each at least one member of the group consisting of a polyvinylpyrrolidone, a sodium citrate, an alkaline inorganic salt, an alginate, a gelatin, a microcrystalline cellulose, a chitosan, a kaolin, a magnesium carbonate, a calcium carbonate, an acacia, a cellulose, a gelatin, a starch, a polyethylene glycol, a polyvinyl alcohol, and a polymethacrylate.

6. The composition of claim 1, wherein the composition comprises the glidant and the lubricant, and wherein the glidant and the lubricant are each at least one member selected from the group consisting of a talc, a silica, a fumed silica, and a colloidal silicon dioxide, and wherein the lubricant is at least one member selected from the group consisting of a talc, a calcium stearate, a sodium stearyl fumarate, a stearic acid, a magnesium stearate, a solid polyethylene glycol, a cocoa butter, a hydrogenated vegetable oil, a mineral oil, a sodium lauryl sulfate, a glyceryl palmitostearate and a glyceryl behenate.

7. The composition of claim 1, wherein the composition comprises the disintegrant, and wherein the disintegrant is at least one member selected from the group consisting of a crospovidone, a crosscarmelose sodium, a sodium starch glycolate, a partially gelatinized starch, and a polacrilin potassium.

8. The composition of claim 1, wherein the composition comprises the surfactant, and wherein the surfactant is at least one member selected from the group consisting of a polysorbate a sodium lauryl sulfate, a lauryl dimethyl amine oxide, a cetyltrimethylammonium bromide, a polyethoxylated alcohol, a polyoxyethylene sorbitan, octoxynol, a n,n-dimethyldodecylamine-n-oxide, a hexadecyltrimethylammonium bromide, a polyoxyl 10 lauryl ether, BRIJ 721, a bile salt, a polyoxyl castor oil, a nonylphenol ethoxylate, a cyclodextrin, a lecithin, and a methylbenzethonium chloride.

9. A pharmaceutical composition in unit dosage form that comprises:
a levothyroxine sodium in one amount selected from the group consisting of 25 μg, 50 μg, 75 μg, 88 μg, 100 μg, 112 μg, 137 μg, 150 μg, 175 μg, 200 μg, and 300 μg;
from 0.001% to 1% w/w of an antioxidant selected from the group consisting of a butylated hydroxyanisole, a vitamin E (alpha tocopherol), a butylated hydroxytoluene, an ascorbic acid, a sodium ascorbate, a sodium bisulfate, and a sodium metabisulfite, and a combination thereof,
from 25% to 95% w/w of a microcrystalline cellulose, and
from 35% to 45% w/w of a calcium phosphate,
wherein the composition is:
free of: added alditol, added monosaccharide, added disaccharide, and added oligosaccharide, and storage stable.

10. The composition of claim 9, wherein the calcium phosphate is a dicalcium phosphate.

11. The composition of claim 9, further comprising from 0.10% to 3% w/w of a polyvinylpyrrolidone.

12. The composition of claim 11, wherein the polyvinylpyrrolidone is a low peroxide K30 polyvinylpyrrolidone.

13. The composition of claim 9, further comprising from 0.1% to 5% w/w of each of a glidant and a lubricant, wherein each of the glidant and the lubricant are at least one member selected from the group consisting of a talc, a silica, a fumed silica, and a colloidal silicon dioxide, and wherein the lubricant lubricant is at least one member selected from the group consisting of a talc, a calcium stearate, a sodium stearyl fumarate, a stearic acid, a magnesium stearate, a solid polyethylene glycol, a cocoa butter, a hydrogenated vegetable oil, a mineral oil, a sodium lauryl sulfate, a glyceryl palmitostearate, and a glyceryl behenate.

14. The composition of claim 13, wherein the glidant is the colloidal silicon dioxide, and wherein the lubricant is the magnesium stearate.

15. The composition of claim 9, further comprising from 0.01% to 5.0% w/w of a surfactant wherein the surfactant is at least one member selected from the group consisting of a polysorbate, a sodium lauryl sulfate, a lauryl dimethyl amine oxide, a cetyltrimethylammonium bromide, a polyethoxylated alcohol, a polyoxyethylene sorbitan, an octoxynol, a n,n-dimethyldodecylamine-n-oxide, a hexadecyltrimethylammonium bromide, a polyoxyl 10 lauryl ether, a BRIJ 721, a bile salt, a polyoxyl castor oil, a nonylphenol ethoxylate, a cyclodextrin a lecithin, and a methylbenzethonium chloride.

16. The composition of claim 15, wherein the surfactant is sodium lauryl sulfate.

20. A pharmaceutical composition in unit dosage form that comprises:
levothyroxine sodium in one amount selected from the group consisting of 25 μg, 50 μg, 75 μg, 88 μg, 100 μg, 112 μg, 137 μg, 150 μg, 175 μg, 200 μg, and 300 μg;
from 0.01% to 0.05% w/vi of a butylated hydroxyanisole;
from 80% to 85% w/w of a microcrystalline cellulose;
from 40% to 45% w/w of a dicalcium phosphate;
from 1% to 3% w/w of a low peroxide K30 polyvinylpyrrolidone;
from 40% to 45% w/w of a colloidal silicon dioxide;
from 40% to 45% w/w of a sodium lauryl sulfate; and
from 40% to 45% w/w of a magnesium stearate,
wherein the composition is free of added alditol, added monosaccharide, added disaccharide, and added oligosaccharide, and wherein the composition is storage stable.
